(19) United States
(12) Patent Application Publication
(10) Pub. No.: US 2007/0099878 A1
(43) Pub. Date: May 3, 2007

(54) Use of porphyrin synthesis substances for carrying out phototherapy and for curing skin and articulation diseases

(75) Inventors: Gerhard Saalmann, Herford (DE); Peter Saalmann, Herford (DE); Hagen Tronnier, Herdecke (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Gerhard SAALMANN, Herford (DE)

(21) Appl. No.: 10/542,355

(22) PCT Filed: Dec. 23, 2003
(86) PCT No.: PCT/DE03/04254

§ 371 Date: Nov. 10, 2006

(30) Foreign Application Priority Data
Jan. 17, 2003 (DE) 103 01 917.0

(51) Int. Cl. A61K 31/60; A61K 31/195

(52) U.S. Cl. 514/159; 514/561

(57) ABSTRACT

The invention relates to the use of porphyrin synthesis substances, in particular 5-amonolevulinic acid, if necessary in combination with salicylates in the form of acetylsalicyc acid and with antioxydising agents in the form of ascorbic acid when a phototherapy is carried out with a light emission whose wavelength ranges from 400 to 700 nm pour treating inflammatory processes, for instance on the skin and/or articulations of mammalians and human beings, in particular for curing arthritis, simple psoriasis, arthopathic psoriasis and neuropathies such as a carpal tunnel syndrome or Bechterev's disease.

The invention relates to the use of substances of the porphyrin synthesis, if desired in combination with salicylates and antioxidants, in the application of phototherapy for treatment of psoriasis or inflammatory processes, such as those of the skin and/or joints of mammals and humans.

The invention further relates to the use of substances of the porphyrin synthesis, if desired in combination with salicylates and antioxidants, for the production of pharmaceuticals for application in phototherapy for treatment of psoriasis or inflammatory processes, such as those of the skin and/or joints of mammals and humans.

Within the scope of the invention, arthritic psoriasis and non-psoriatic polyarthritis in particular will be understood as inflammatory processes.

In contrast to non-psoriatic polyarthritis, such as rheumatoid arthritis and similar syndromes, arthritic psoriasis is the simultaneous occurrence of plaque psoriasis and monoarthritic or polyarthritic joint changes, which affect in particular the finger, ankle and toe joints as well as the spinal column and hip joints, not to mention other joints. In arthritic psoriasis patients, the serological tests for rheumatoid condition are usually negative, in contrast to the results in rheumatoid arthritis. At present, psoriatic arthritis is treated predominantly with non-steroidal anti-inflammatory drugs, although gold preparations, glucocorticosteroids and retinoids as well as methotrexate and cyclosporins are also used. The success of these drug treatments is often unsatisfactory, however, and in particular is associated with relatively strong adverse reactions or risks of adverse reactions. In particular, undesired adverse reactions are observed in long-term therapy, which is usually necessary.

The object of the invention is to largely prevent such adverse reactions by providing and administering active ingredients or combinations thereof having weak adverse reactions in conjunction with subsequent irradiation with visible light, and at the same time to considerably increase the success rate and tolerance compared with the known treatment methods based purely on medications.

Phototherapeutic measures in combination with the application of medications are already known from diverse sources:

For example, German Patent A 10063076 describes the use of aminolevulinic acid for prevention of re-stenosis in photodynamic therapy with application of sublethal light doses.

It is also already known that various active ingredients in conjunction with irradiation of the body with UV or VIS light are effective for the treatment of skin diseases (Psoriasis, Medizin in der Praxis [Medicine in Practice], 20/00, pp. 55-59).

From Mund-Kiefer-und Gesichtschirurgie [Oral and Facial Surgery], Abstract Volume 5, Issue 2 (2001), pp. 98-101, ISSN No.: 1432-9417, there is also known an experimental 5-aminolevulinic acid-induced photodynamic therapy (ALA-PDT) for the treatment of solid tumors. Laser light with a wavelength of 635 nm and a power of 0.75 watt has been used as the light source.

It is further known that aspirin together with non-steroidal anti-inflammatory drugs such as ibuprofen can be used for treatment of arthritis (Medications for Arthritis: www.orthop.washington.edu/arthritis/medications/05).

To achieve the object of the invention, it is proposed that substances of the porphyrin synthesis as well as the pharmacologically usable esters or salts thereof with pharmacologically compatible acids or bases, if desired in combination with salicylates, preferably acetylsalicylic acid and possibly compatible antioxidants, preferably ascorbic acid, be prepared and used for radiation therapy with light having a wavelength of 400 to 700 nm, preferably 520 to 580 nm, especially in the range of 545 nm, for the treatment of psoriasis and/or inflammatory changes in joints of humans or mammals.

The prior art methods cited in the foregoing do not contribute to the proposed achievement of the object of the invention and do not disclose to the person skilled in the art any way in which psoriasis or inflammatory processes in the joints can be effectively treated successfully and reliably while simultaneously excluding adverse reactions to the greatest extent.

The subject matter of the invention is therefore the use, characterized in more detail in the claims, of substances of the porphyrin synthesis, especially 5-aminolevulinic acid, if desired in combination with salicylates and antioxidants.

The substances of the porphyrin synthesis (preferably 5-aminolevulinic acid, abbreviated as ALA) as well as the pharmacologically usable derivatives or salts thereof are used either alone or in combination with salicylates (preferably acetylsalicylic acid). If desired, they are additionally combined with antioxidants, preferably ascorbic acid.

The inventive substances can be administered systemically or locally, parenterally or enterally, preferably orally or topically in the form of conventional pharmaceutical preparations. The active ingredients or combinations thereof chosen in each case can be taken orally in particularly simple manner by the patient, for example dissolved or suspended in water or fruit juice. Special injectable forms can be provided in particular for local treatments.

For local treatment, it is advantageous to administer the active ingredients or combinations thereof either topically by percutaneous infiltration into the tissue of the affected body part or to inject them deeper into the affected tissue. Conceivable options are therefore percutaneous application forms such as salves, creams or lotions on the one hand, as are sterile solutions or emulsions suitable for parenteral injection on the other hand. For salve bases, it is recommended that an occlusive dressing coated with the active ingredients be applied before irradiation, thus both increasing the efficacy and shortening the necessary exposure time.

Consequently, all standard pharmaceutical forms suitable for parenteral or enteral administration and in particular for oral or possibly even topical administration are conceivable as typical pharmaceutical formulations. Examples include powders, tablets, coated pills, soft or hard gelatin capsules, effervescent tablets, emulsions, oils, solutions or lyophilized substances, as well as sterile injection solutions or emulsions containing standard auxiliary and adjuvant substances.

By virtue of the novel combination therapy according to the present invention, there is surprisingly achieved a reduction of acute or chronic, specific or unspecific joint inflammations that is at least partial but often is even complete, as well as the disappearance of mobility restrictions, extensive freedom from pain and recovery of swellings to the normal condition of the afflicted body regions.

Similarly, the therapy proposed according to the invention is particularly effective, in its different clinical forms, for treatment of skin psoriasis. The novel combination of the administration of active ingredients having weak adverse reactions with irradiation using light of a defined wavelength region from 400 to 700 nm, preferably 520 to 580 nm, especially in the region around 545 nm, is indicated in particular for the treatment of arthritic psoriasis (psoriatic arthritis), of arthritis forms having another pathogenesis, of neuropathies (such as carpal tunnel syndrome) and of ankylosing spondylarthritis (Bekhterev's disease).

According to the invention, 5-aminolevulinic acid (ALA) or esters thereof, such as the methyl ester (MALA) or salts thereof, especially the hydrochlorides, are to be understood in particular as substances of the porphyrin synthesis.

In general, there can be used all substances that can be metabolized to protoporphyrin IX (PP IX) in human or animal tissue during treatment, since PP IX is the effective photosensitizer during irradiation; this is then further converted to heme in the organism.

Within the scope of the present invention, the esters of carboxyl groups of the active ingredient being used with saturated or unsaturated, straight-chain or branched C¹ to C⁴ aliphatic or C³ to C⁷ cycloaliphatic alcohols or other compounds containing an alcoholic OH group that are safe or support the therapy are understood by the term esters. Conversely, esters with alcoholic OH groups of active ingredients formed with pharmacologically safe acids such as acetic acid or propionic acid are naturally also conceivable.

These alcohols include in particular C¹ to C⁴ aliphatic alcohols such as methanol, ethanol, propanol and isopropanol.

Within the scope of the invention, salts with pharmacologically compatible inorganic or organic acids or bases are understood as salt components with basic or acid groups of the substances used according to the invention. Examples include hydrochlorides and hydrobromides and, by analogy, the sulfates, phosphates, nitrates, acetates, propionates, citrates, lactates, mandelates, sorbates, ascorbates or maleates.

With acid groups, especially carboxyl groups of the active ingredients, there are obtained usable lithium, sodium, potassium, calcium, magnesium or zinc salts, as well as quaternary ammonium salts with ammonia or aliphatic amines such as methylamine or ethylamine. It is understood that very many further salt components, as are already widely employed and known for pharmaceuticals, are also conceivable here.

Acetylsalicylic acid, which has weak adverse reactions, is usable in particular as the salicylate. Other possibilities are salicylic acid itself or other active salicylic acid derivatives or salts thereof, such as sodium salicylate, methyl salicylate or hydroxyethyl salicylate.

As antioxidants there can be used all compounds having an adequate redox potential that are pharmacologically safe and that if necessary support the therapy, especially ascorbic acid. Other examples also include the following substances or salts or derivatives thereof: isoascorbic acid, tocopherol, gluconic acid or carotenoids.

A combination of 5-aminolevulinic acid with acetylsalicylic acid and if desired ascorbic acid in the weight ratio of approximately 1:3:2 has proved effective.

The course of therapy comprises parenteral or enteral, especially oral or topical administration of the inventive formulations, followed by a waiting time of 60 to 180, preferably 150 minutes and subsequent phototherapy with irradiation doses at non-cytotoxic levels in the wavelength region indicated hereinabove. Not only is whole-body irradiation effective, but also partial areas and individual joints can be irradiated particularly effectively. It is also possible, by means of optical fibers or endoscopes, to guide the light directly to the inflamed tissue.

An effective radiation dose in the range of approximately 5 to 50 J/cm² is considered to be non-cytotoxic. It must be selected as a function of the sensitivity of the patient, so that visible and undesired secondary phenomena such as skin irritations or signs of inflammation of the irradiated body regions are prevented. Since the therapy usually consists of several, preferably 6 to 15 irradiations, it is easily possible for the treating physician to adjust to the optimal irradiation dose and thus to avoid overdoses. Since the irradiation takes place with visible light, which is not very aggressive, it is in any case unproblematic within very wide limits compared with the treatment using ultraviolet light.

The exposure and irradiation system can be composed of one or more lamp units, by which the skin is irradiated completely or partly with visible light of the wavelength region indicated hereinabove, quite particularly preferably with green light in the wavelength range of 540 to 550 nm. The intensity of the treatment is controlled as a function of the patient's constitution and of the duration and seriousness of the disease, by variation of the active ingredients, the irradiation intensity, the wavelength, the irradiation distance, the irradiation duration and, in the case of repeated treatments, the time interval between irradiations. The necessary irradiation dose or irradiation duration can be directly determined by the physician on the basis of the above criteria and of the special medical history.

According to the invention, an irradiation dose of 5 to 50 J/cm² is proposed for whole-body irradiation. An irradiation dose of approximately 15 J/cm² is preferred. For local treatment, an irradiation dose of 10 to 80 J/cm² is recommended. The irradiation duration depends on the distance of the radiation source from the body surface to be irradiated and the radiated power of the source used. In the normal case, the light sources for whole-body irradiation should be at a distance of 10 to 50 cm. For a radiated power of 20 mW/cm², the irradiation time per treatment is approximately 20 to 30 minutes.

For local treatment, the distance of a source having a power of 40 mW/cm² from the surface of the body part to be treated is approximately 10 to 15 cm. In this case the irradiation duration is between 10 and 20 minutes. The cited parameters relate to the normal case, and it is entirely possible to use different values within the scope of what can be tolerated.

### Study Results

Five patients diagnosed with severe arthritic psoriasis were treated in a pilot study.

The results are summarized as follows:

| | Mean age | Mean duration of | |
|---|---|---|---|
| Sex | (years) | disease (years) | Previous treatment |
| m: 1 | 50.4 | 16.4 | MTX, corticosteroids, |
| f: 4 | | | non-steroidal anti- |
| | | | inflammatory drugs |

Results after three weeks of therapy (9 applications at equal intervals)

| | |
|---|---|
| Very good: | 4 (free of symptoms) |
| Good: | 1 (mild pains and mobility restrictions) |
| Moderate or no improvement: | 0 |

### CASE EXAMPLE

A 41-year-old male with a body weight of 80 kg had been suffering for 15 years from erythematosquamous psoriasis of the most common sites and for 5 years from arthritic psoriasis of the interphalangeal joints of the hands and feet. He was complaining about mobility restriction, morning stiffness and pain upon application of pressure. The previous therapy consisted of the administration of non-steroidal anti-inflammatory drugs and methotrexate in a dosage of 15 mg/week as antirheumatic. The treatment success was moderate.

Two weeks after discontinuation of the previous therapy, a combination of 160 mg (2 mg/kg body weight) of 5-aminolevulinic acid, 400 mg (5 mg/kg body weight) of acetylsalicylic acid and 240 mg (3 mg/kg body weight) of ascorbic acid was administered orally three times per week over a period of three weeks. In each case, whole-body irradiation with green light (wavelength 540 to 550 nm, dose: 15 J/cm²) was undertaken after a waiting time of 150 minutes following the drug application. The result of the treatment was good. Both the morning stiffness and the pains abated considerably. Compared with the result of the previous treatment, a clear decrease of the subjective and objective symptoms was achieved without subjective adverse reactions. The arthritis score (improvement in %) was 56, and that of the morning stiffness was 83%. The laboratory values (transaminases, blood counts, erythrocyte sedimentation rate) remained unchanged.

1. The use of substances of the porphyrin synthesis or pharmacologically compatible salts thereof for the production of pharmaceuticals for application of phototherapy with light having a wavelength of 400 to 700 nm, as well as for the treatment of psoriasis and of inflammatory processes of the skin and/or joints of mammals and humans.

2. The use according to claim 1, characterized in that a salicylate is additionally used as the active ingredient.

3. The use according to claim 2, characterized in that an antioxidant is additionally used as the active ingredient.

4. The use according to claims 1 to 3, characterized in that 5-aminolevulinic acid (ALA) is used as the substance of the porphyrin synthesis.

5. The use according to claims 1 to 4, characterized in that acetylsalicylic acid is used as the salicylate.

6. The use according to claims 1 to 5, characterized in that ascorbic acid or a pharmacologically compatible salt thereof is used as the antioxidant.

7. The use according to claims 1 to 6, characterized in that the wavelength of the light is 400 to 700 nm.

8. The use according to claim 7, characterized in that the wavelength of the light is approximately 545 nm.

9. The use according to claims 1 to 8 for the treatment of arthritis, plaque psoriasis, arthritic psoriasis, neuropathies such as carpal tunnel syndrome or Bekhterev's disease.

10. The use of substances of the porphyrin synthesis according to claims 1 to 9 for phototherapy.
